(19) United States
(12) Patent Application Publication
(10) Pub. No.: US 2015/0374978 A1
(43) Pub. Date: Dec. 31, 2015

(54) METHODS AND SYSTEMS FOR ELECTRICAL STIMULATION INCLUDING A SHIELDED LEAD

(71) Applicant: Boston Scientific Neuromodulation Corporation, Valencia, CA (US)

(72) Inventors: Joshua Dale Howard, Winnetka, CA (US); David Ernest Wechter, Santa Clarita, CA (US)

(21) Appl. No.: 14/748,127

(22) Filed: Jun. 23, 2015

Related U.S. Application Data
(60) Provisional application No. 62/018,283, filed on Jun. 27, 2014.

(51) Int. Cl. A61N 1/08; A61N 1/05

(52) U.S. Cl. CPC A61N 1/08; A61N 1/05

(57) ABSTRACT

An electrical stimulation lead includes at least one lead body having a distal end portion, a proximal end portion, and an outer surface. Each lead body has a lead jacket. The lead also includes electrodes disposed along the distal end portion of the at least one lead body; terminals disposed along the proximal end portion of the at least one lead body; and conductors extending within the at least one lead body to electrically couple the terminals to the electrodes. The conductors include at least one first conductor and at least one second conductor with the at least one first conductor coiled around the at least one second conductor. The lead further includes a conductive RF shield disposed between at least a portion of the lead jacket and around at least a portion of each of the conductors. A lead extension can be similarly constructed.

### CROSS-REFERENCE TO RELATED APPLICATIONS

This application claims the benefit under 35 U.S.C. §119(e) of U.S. Provisional Patent Application Ser. No. 62/018,283, filed Jun. 27, 2014, which is incorporated herein by reference.

### FIELD

The present invention is directed to the area of implantable electrical stimulation systems and methods of making and using the systems. The present invention is also directed to implantable electrical stimulation leads having an RF shield, as well as to methods of making and using the leads and electrical stimulation systems.

### BACKGROUND

Implantable electrical stimulation systems have proven therapeutic in a variety of diseases and disorders. For example, spinal cord stimulation systems have been used as a therapeutic modality for the treatment of chronic pain syndromes. Peripheral nerve stimulation has been used to treat chronic pain syndrome and incontinence, with a number of other applications under investigation. Functional electrical stimulation systems have been applied to restore some functionality to paralyzed extremities in spinal cord injury patients.

Stimulators have been developed to provide therapy for a variety of treatments. A stimulator can include a control module (with a pulse generator), one or more leads, and an array of stimulator electrodes on each lead. The stimulator electrodes are in contact with or near the nerves, muscles, or other tissue to be stimulated. The pulse generator in the control module generates electrical pulses that are delivered by the electrodes to body tissue.

### BRIEF SUMMARY

One embodiment is an electrical stimulation lead including at least one lead body having a distal end portion, a proximal end portion, and an outer surface. Each lead body has a lead jacket forming at least a portion of the outer surface of the lead body and including a non-conductive lead jacket material. The lead also includes electrodes disposed along the distal end portion of the at least one lead body; terminals disposed along the proximal end portion of the at least one lead body; and conductors extending within the at least one lead body to electrically couple the terminals to the electrodes. The conductors include at least one first conductor and at least one second conductor with the at least one first conductor coiled around the at least one second conductor. The lead further includes a conductive RF shield disposed between at least a portion of the lead jacket and around at least a portion of each of the conductors.

Another embodiment is a lead extension including at least one body having a distal end portion, a proximal end portion, and an outer surface. Each body has a jacket forming at least a portion of the outer surface of the body and including a non-conductive jacket material. The lead extension also includes terminals disposed along the proximal end portion of the at least one body and a connector disposed along the distal end portion of the at least one body to receive a portion of a lead. The connector includes connector contacts. The lead extension further includes conductors extending within the at least one body from the plurality of connector contacts to the plurality of terminals. The conductors are divided into one or more first conductors and one or more second conductors with the one or more first conductors coiled around the one or more second conductors. The lead extension also includes a conductive RF shield disposed between at least a portion of the jacket and around at least a portion of each of the conductors.

In yet another embodiment, an electrical stimulation system includes one or both of the lead or lead extension described above, as well as a control module coupleable to the lead or lead extension.

In at least some embodiments of the lead or lead extension, the RF shield is a conductive coiled tube or a conductive braided tube. In at least some embodiments of the lead or lead extension, the one or more second conductors are also coiled. In at least some embodiments of the lead or lead extension, the one or more second conductors are disposed in a multi-lumen conductor guide. In at least some embodiments of the lead or lead extension, the conductive RF shield is configured and arranged to be electrically floating.

In at least some embodiments of the lead or lead extension, the at least one first conductor is a single coiled first conductor and the at least one second conductor is multiple second conductors. In at least some of these embodiments, the lead or lead extension also includes a multi-lumen conductor guide disposed within the lead body and within the single coiled first conductor where the multi-lumen conductor guide includes conductor lumens with the second conductors disposed within the conductor lumens. In at least some of these embodiments, the second conductors extend straight relative to the lead body.

In at least some embodiments of the lead or lead extension, the at least one first conductor is multiple first conductors cowound around the at least one second conductor. In at least some of these embodiments, the at least one second conductor is coiled. In at least some of these embodiments, the at least one first conductor and the at least one second conductor are coiled in a same direction. In other embodiments, the at least one first conductor and the at least one second conductor are coiled in opposite directions.

In at least some embodiments of the lead or lead extension, the RF shield extends between the terminals and the electrodes. In at least some embodiments of the lead or lead extension, the RF shield is disposed between the lead jacket and the conductors.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic view of one embodiment of an electrical stimulation system that includes a paddle lead electrically coupled to a control module, according to the invention;

FIG. 2 is a schematic view of one embodiment of an electrical stimulation system that includes a percutaneous lead electrically coupled to a control module, according to the invention;

FIG. 3A is a schematic view of one embodiment of the control module of FIG. 1 configured and arranged to electrically couple to an elongated device, according to the invention;

FIG. 3B is a schematic view of one embodiment of a lead extension configured and arranged to electrically couple the elongated device of FIG. 2 to the control module of FIG. 1, according to the invention;

FIG. 4A is a schematic cross-sectional view of one embodiment of a lead with a RF shield, according to the invention;

FIG. 4B is a schematic cross-sectional view of another embodiment of a lead with a RF shield, according to the invention;

FIG. 5 is a schematic perspective view of one embodiment of an arrangement of conductors for an electrical stimulation lead, according to the invention;

FIG. 6 is a schematic perspective view of a second embodiment of an arrangement of conductors for an electrical stimulation lead, according to the invention;

FIG. 7 is a schematic perspective view of a third embodiment of an arrangement of conductors for an electrical stimulation lead, according to the invention;

FIG. 8 is a schematic perspective view of a fourth embodiment of an arrangement of conductors for an electrical stimulation lead, according to the invention; and

FIG. 9 is a schematic overview of one embodiment of components of a stimulation system, including an electronic subassembly disposed within a control module, according to the invention.

### DETAILED DESCRIPTION

The present invention is directed to the area of implantable electrical stimulation systems and methods of making and using the systems. The present invention is also directed to implantable electrical stimulation leads having an RF shield, as well as to methods of making and using the leads and electrical stimulation systems.

Suitable implantable electrical stimulation systems include, but are not limited to, at least one lead with one or more electrodes disposed along a distal end of the lead and one or more terminals disposed along the one or more proximal ends of the lead. Leads include, for example, percutaneous leads, paddle leads, and cuff leads. Examples of electrical stimulation systems with leads are found in, for example, U.S. Pat. Nos. 6,181,969; 6,516,227; 6,609,029; 6,609,032; 6,741,892; 7,949,395; 7,244,150; 7,672,734; 7,761,165; 7,974,706; 8,175,710; 8,224,450; and 8,364,278; and U.S. Patent Application Publication No. 2007/0150036, all of which are incorporated by reference.

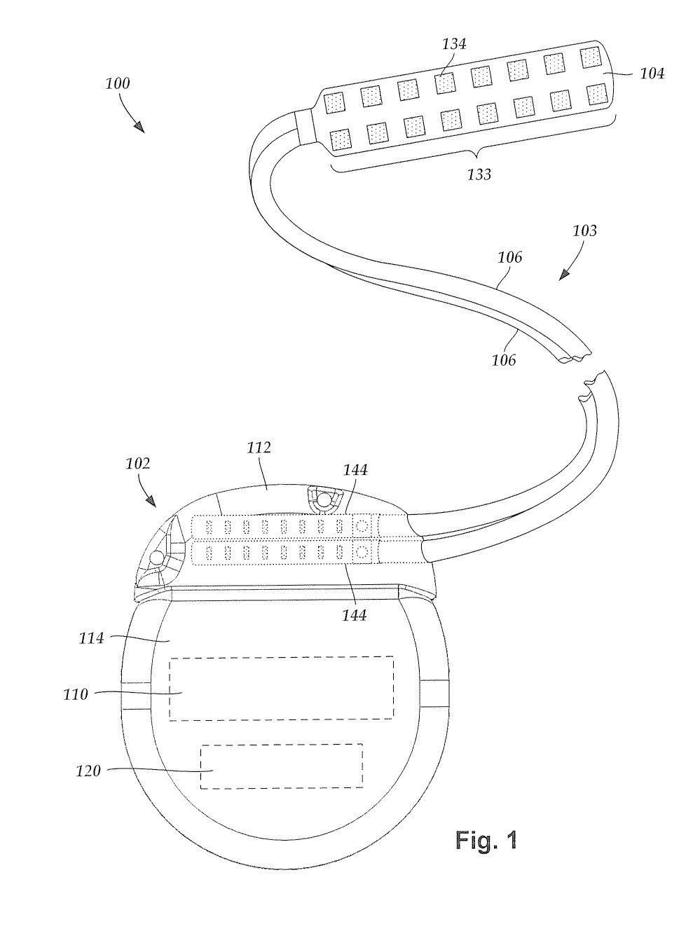

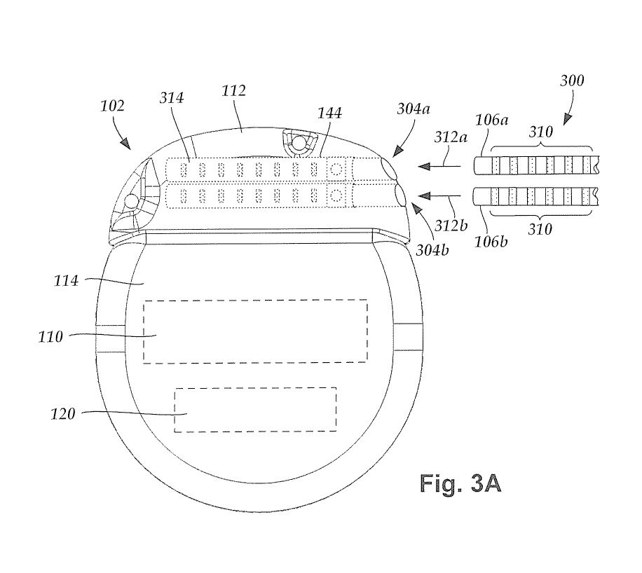

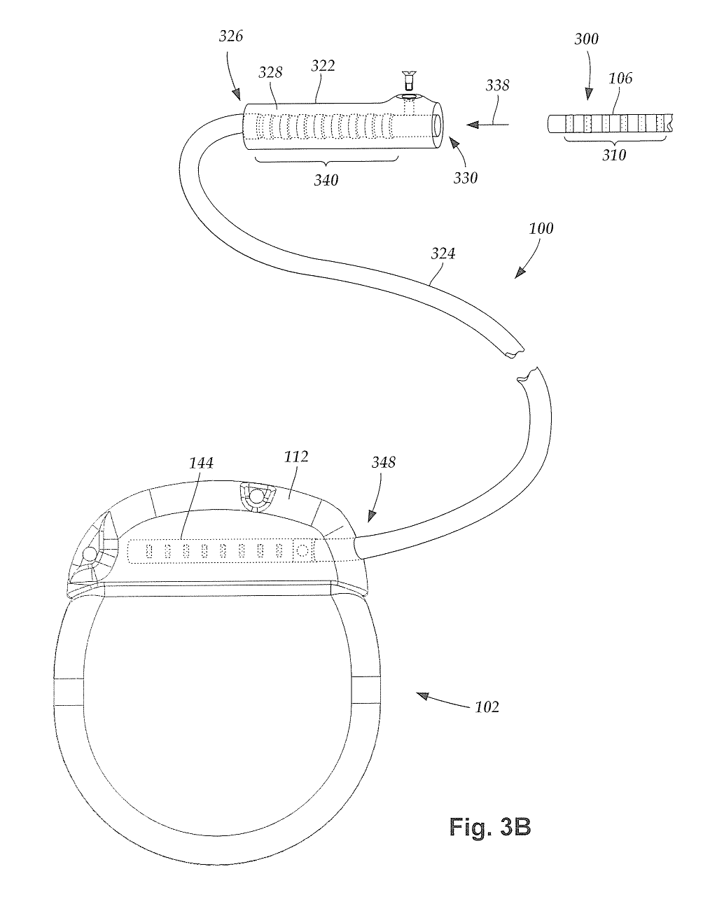

FIG. 1 illustrates schematically one embodiment of an electrical stimulation system 100. The electrical stimulation system includes a control module (e.g., a stimulator or pulse generator) 102 and a lead 103 coupleable to the control module 102. The lead 103 includes a paddle body 104 and one or more lead bodies 106. In FIG. 1, the lead 103 is shown having two lead bodies 106. It will be understood that the lead 103 can include any suitable number of lead bodies including, for example, one, two, three, four, five, six, seven, eight or more lead bodies 106. An array 133 of electrodes, such as electrode 134, is disposed on the paddle body 104, and an array of terminals (e.g., 310 in FIG. 3A-3B) is disposed along each of the one or more lead bodies 106.

It will be understood that the electrical stimulation system can include more, fewer, or different components and can have a variety of different configurations including those configurations disclosed in the electrical stimulation system references cited herein. For example, instead of a paddle body, the electrodes can be disposed in an array at or near the distal end of a lead body forming a percutaneous lead.

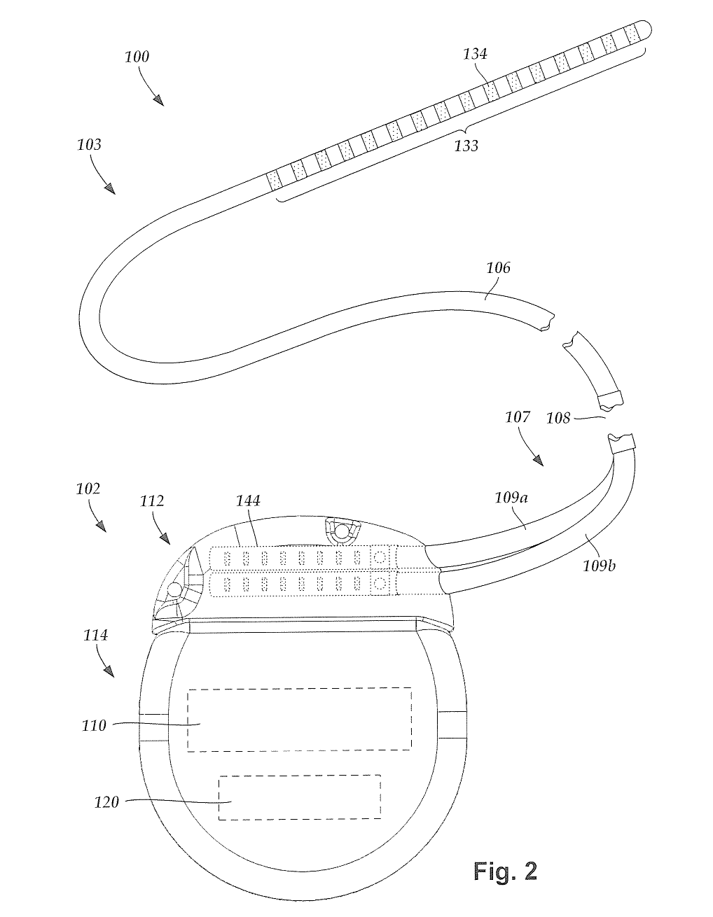

FIG. 2 illustrates schematically another embodiment of the electrical stimulation system 100, where the lead 103 is a percutaneous lead. In FIG. 2, the electrodes 134 are shown disposed along the one or more lead bodies 106. In at least some embodiments, the lead 103 is isodiametric along a longitudinal length of the lead body 106.

The lead 103 can be coupled to the control module 102 in any suitable manner. In FIG. 1, the lead 103 is shown coupling directly to the control module 102. In at least some other embodiments, the lead 103 couples to the control module 102 via one or more intermediate devices (324 in FIG. 3B). For example, in at least some embodiments one or more lead extensions 324 (see e.g., FIG. 3B) can be disposed between the lead 103 and the control module 102 to extend the distance between the lead 103 and the control module 102. Other intermediate devices may be used in addition to, or in lieu of, one or more lead extensions including, for example, a splitter, an adaptor, or the like or combinations thereof. It will be understood that, in the case where the electrical stimulation system 100 includes multiple elongated devices disposed between the lead 103 and the control module 102, the intermediate devices may be configured into any suitable arrangement.

In FIG. 2, the electrical stimulation system 100 is shown having a splitter 107 configured and arranged for facilitating coupling of the lead 103 to the control module 102. The splitter 107 includes a splitter connector 108 configured to couple to a proximal end of the lead 103, and one or more splitter tails 109a and 109b configured and arranged to couple to the control module 102 (or another splitter, a lead extension, an adaptor, or the like).

With reference to FIGS. 1 and 2, the control module 102 typically includes a connector housing 112 and a sealed electronics housing 114. An electronic subassembly 110 and an optional power source 120 are disposed in the electronics housing 114. A control module connector 144 is disposed in the connector housing 112. The control module connector 144 is configured and arranged to make an electrical connection between the lead 103 and the electronic subassembly 110 of the control module 102.

The electrical stimulation system or components of the electrical stimulation system, including the paddle body 104, the one or more of the lead bodies 106, and the control module 102, are typically implanted into the body of a patient. The electrical stimulation system can be used for a variety of applications including, but not limited to deep brain stimulation, neural stimulation, spinal cord stimulation, muscle stimulation, and the like.

The electrodes 134 can be formed using any conductive, biocompatible material. Examples of suitable materials include metals, alloys, conductive polymers, conductive carbon, and the like, as well as combinations thereof. In at least some embodiments, one or more of the electrodes 134 are formed from one or more of: platinum, platinum iridium, palladium, palladium rhodium, or titanium.

Any suitable number of electrodes 134 can be disposed on the lead including, for example, four, five, six, seven, eight, nine, ten, eleven, twelve, fourteen, sixteen, twenty-four, thirty-two, or more electrodes 134. In the case of paddle leads, the electrodes 134 can be disposed on the paddle body 104 in any suitable arrangement. In FIG. 1, the electrodes 134 are arranged into two columns, where each column has eight electrodes 134.

The electrodes of the paddle body 104 (or one or more lead bodies 106) are typically disposed in, or separated by, a non-conductive, biocompatible material such as, for example, silicone, polyurethane, polyetheretherketone (“PEEK”), epoxy, and the like or combinations thereof. The one or more lead bodies 106 and, if applicable, the paddle body 104 may be formed in the desired shape by any process including, for example, molding (including injection molding), casting, and the like. The non-conductive material typically extends from the distal ends of the one or more lead bodies 106 to the proximal end of each of the one or more lead bodies 106.

In the case of paddle leads, the non-conductive material typically extends from the paddle body 104 to the proximal end of each of the one or more lead bodies 106. Additionally, the non-conductive, biocompatible material of the paddle body 104 and the one or more lead bodies 106 may be the same or different. Moreover, the paddle body 104 and the one or more lead bodies 106 may be a unitary structure or can be formed as two separate structures that are permanently or detachably coupled together.

Terminals (e.g., 310 in FIGS. 3A-3B) are typically disposed along the proximal end of the one or more lead bodies 106 of the electrical stimulation system 100 (as well as any splitters, lead extensions, adaptors, or the like) for electrical connection to corresponding connector contacts (e.g., 314 in FIG. 3A). The connector contacts are disposed in connectors (e.g., 144 in FIGS. 1-3B; and 322 FIG. 3B) which, in turn, are disposed on, for example, the control module 102 (or a lead extension, a splitter, an adaptor, or the like). Electrically conductive wires, cables, or the like (not shown) extend from the terminals to the electrodes 134. Typically, one or more electrodes 134 are electrically coupled to each terminal. In at least some embodiments, each terminal is only connected to one electrode 134.

The electrically conductive wires (“conductors”) may be embedded in the non-conductive material of the lead body 106 or can be disposed in one or more lumens (not shown) extending along the lead body 106. In some embodiments, there is an individual lumen for each conductor. In other embodiments, two or more conductors extend through a lumen. Other arrangements of the conductors are described below.

There may also be one or more lumens (not shown) that open at, or near, the proximal end of the one or more lead bodies 106, for example, for inserting a stylet to facilitate placement of the one or more lead bodies 106 within a body of a patient. Additionally, there may be one or more lumens (not shown) that open at, or near, the distal end of the one or more lead bodies 106, for example, for infusion of drugs or medication into the site of implantation of the one or more lead bodies 106. In at least one embodiment, the one or more lumens are flushed continually, or on a regular basis, with saline, epidural fluid, or the like. In at least some embodiments, the one or more lumens are permanently or removably sealable at the distal end.

FIG. 3A is a schematic side view of one embodiment of a proximal end of one or more elongated devices 300 configured and arranged for coupling to one embodiment of the control module connector 144. The one or more elongated devices may include, for example, one or more of the lead bodies 106 of FIG. 1, one or more intermediate devices (e.g., a splitter, the lead extension 324 of FIG. 3B, an adaptor, or the like or combinations thereof), or a combination thereof.

The control module connector 144 defines at least one port into which a proximal end of the elongated device 300 can be inserted, as shown by directional arrows 312a and 312b. In FIG. 3A (and in other figures), the connector housing 112 is shown having two ports 304a and 304b. The connector housing 112 can define any suitable number of ports including, for example, one, two, three, four, five, six, seven, eight, or more ports.

The control module connector 144 also includes a plurality of connector contacts, such as connector contact 314, disposed within each port 304a and 304b. When the elongated device 300 is inserted into the ports 304a and 304b, the connector contacts 314 can be aligned with a plurality of terminals 310 disposed along the proximal end(s) of the elongated device(s) 300 to electrically couple the control module 102 to the electrodes (134 of FIG. 1) disposed on the paddle body 104 of the lead 103. Examples of connectors in control modules are found in, for example, U.S. Pat. Nos. 7,244,150 and 8,224,450, which are incorporated by reference.

FIG. 3B is a schematic side view of another embodiment of the electrical stimulation system 100. The electrical stimulation system 100 includes a lead extension 324 that is configured and arranged to couple one or more elongated devices 300 (e.g., one of the lead bodies 106 of FIGS. 1 and 2, the splitter 107 of FIG. 2, an adaptor, another lead extension, or the like or combinations thereof) to the control module 102. In FIG. 3B, the lead extension 324 is shown coupled to a single port 304 defined in the control module connector 144. Additionally, the lead extension 324 is shown configured and arranged to couple to a single elongated device 300. In alternate embodiments, the lead extension 324 is configured and arranged to couple to multiple ports 304 defined in the control module connector 144, or to receive multiple elongated devices 300, or both.

A lead extension connector 322 is disposed on the lead extension 324. In FIG. 3B, the lead extension connector 322 is shown disposed at a distal end 326 of the lead extension 324. The lead extension connector 322 includes a connector housing 328. The connector housing 328 defines at least one port 330 into which terminals 310 of the elongated device 300 can be inserted, as shown by directional arrow 338. The connector housing 328 also includes a plurality of connector contacts, such as connector contacts 340. When the elongated device 300 is inserted into the port 330, the connector contacts 340 disposed in the connector housing 328 can be aligned with the terminals 310 of the elongated device 300 to electrically couple the lead extension 324 to the electrodes (134 of FIGS. 1 and 2) disposed along the lead (103 in FIGS. 1 and 2).

In at least some embodiments, the proximal end of the lead extension 324 is similarly configured and arranged as a proximal end of the lead 103 (or other elongated device 300). The lead extension 324 may include a plurality of electrically conductive wires (not shown) that electrically couple the connector contacts 340 to a proximal end 348 of the lead extension 324 that is opposite to the distal end 326. In at least some embodiments, the conductive wires disposed in the lead extension 324 can be electrically coupled to a plurality of terminals (not shown) disposed along the proximal end 348 of the lead extension 324. In at least some embodiments, the proximal end 348 of the lead extension 324 is configured and arranged for insertion into a connector disposed in another lead extension (or another intermediate device). In other embodiments (and as shown in FIG. 3B), the proximal end 348 of the lead extension 324 is configured and arranged for insertion into the control module connector 144.

Conventional electrical stimulation systems may be potentially unsafe for use with magnetic resonance imaging (“MRI”) due to the effects of electromagnetic fields in an MRI environment. A common mechanism for causing the electrical interactions between the electrical stimulation system and RF irradiation is common-mode coupling of the applied electromagnetic fields that act as a series of distributed sources along elongated conductive structures, such as leads or lead extensions, or conductors within leads or lead extensions. Common-mode induced RF currents can reach amplitudes of greater than one ampere in MRI environments. Such currents can cause heating and potentially disruptive voltages within electronic circuits.

Some of the effects of RF irradiation may include, for example, inducing current in the lead or lead extension, causing undesired heating at the electrodes of the lead that may potentially cause tissue damage, undesired or unexpected operation of electronic components, or premature failure of electronic components. Additionally, when an electrical stimulation system is used within an MRI scanner environment, the electrical interactions between the electrical stimulation system and the MRI may cause distortions in images formed by the MRI system.

A lead or lead extension can include a RF shield within the lead body or lead extension body and extending at least partway (or all the way) between, but not including, the distal-most terminal and the proximal-most electrode (for a lead) or connector (for a lead extension).

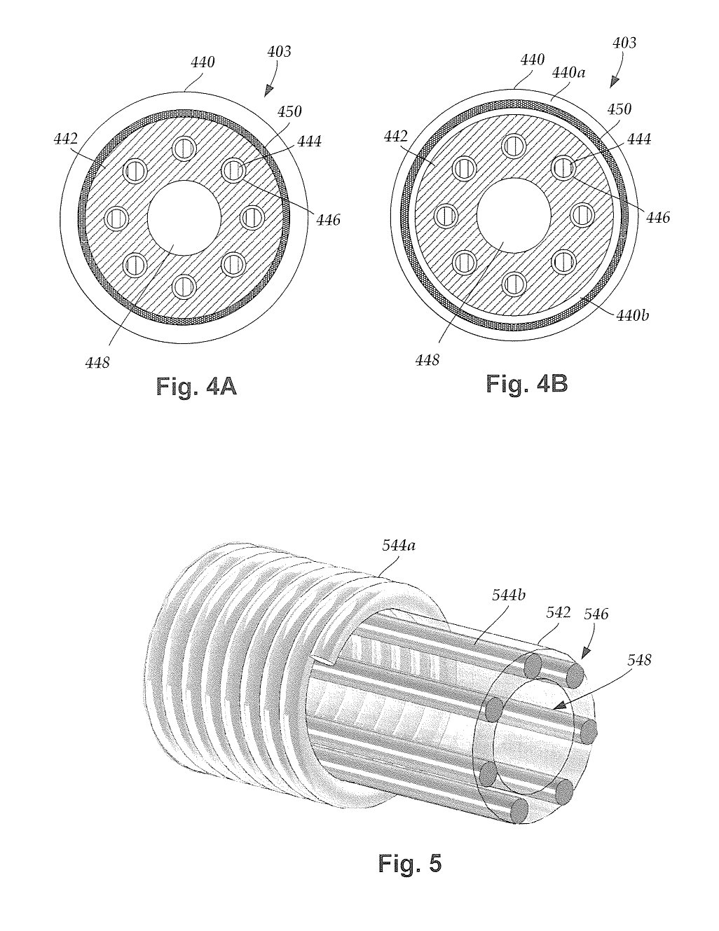

FIG. 4A illustrates one embodiment of a lead 403 that includes a lead jacket 440 and a RF shield 450 disposed over an elongated multi-lumen conductor guide 442 having one or more conductor lumens 446 (preferably, multiple conductor lumens) arranged about a central lumen 448. Conductors 444 are disposed in the conductor lumens 446. It will be understood that although a lead 403 is illustrated in FIG. 4A, the same elements can be used in a lead extension. FIG. 4B illustrates another embodiment in which the RF shield 450 is disposed within the lead jacket 440.

The RF shield 450 of the lead 403 can have any suitable form including, but not limited to, a conductive braided tube or a conductive coiled tube. The RF shield 450 is made of a biocompatible conductive material, such as, for example, platinum, titanium, MP35N, 35N LT, 316L stainless steel, tantalum, or any other suitable metal or alloy.

The RF shield 450 prevents or reduces the induction of current in the conductors 444 of the lead 403 (or lead extension) disposed within the shield when exposed to RF irradiation. In at least some embodiments, the RF shield 450 can be designed to shield the lead 403 from RF at one or more specific frequencies, such as specific MRI frequencies (for example, 64 MHz, 128 MHz, or both) or any other frequency, frequency band, or set of frequencies or frequency bands. For example, the braiding pattern of a braided tube, the pitch of a coiled tube, the diameter of the braided or coiled tube, or any other parameter of the RF shield 450 or any combination of parameters can be selected to shield the conductors 444 within the lead from RF at the specific frequency or frequencies or frequency band(s). The RF shield may also prevent or reduce induction of current arising from other electromagnetic sources, such as changing magnetic fields (for example, the changing magnetic gradient fields of an MRI apparatus.)

The RF shield 450 may extend along the entirety of the lead 403 between, but not including, the electrodes (see electrodes 134 of FIGS. 1 and 2) and the terminals (see terminals 310 of FIGS. 3A and 3B) (or between, but not including, the terminals and connector of a lead extension) or may extend only partway along (for example, at least 95%, 90%, 80%, 75%, 66%, 50%, or 25% of the length of) the lead or lead extension.

The RF shield 450 can be electrically floating so that it has no electrical connection to the control module, lead, or lead extension or to any of the electrodes, terminals, or contacts of the lead, lead extension or control module. Alternatively, the shield may be grounded through the lead, lead extension, or control module through one of the electrodes, terminals, contacts, or through a separate grounding contact. In other embodiments, the RF shield may also act as a conductor between an electrode and a terminal of a lead or between a terminal and a connector contact in the connector of a lead extension.

The lead jacket 440 can be made of any suitable biocompatible material, such as polymeric materials. Examples of materials for the lead jacket include, but are not limited to, polyurethane and silicone. In the embodiment of FIG. 4A, the RF shield 450 is between the lead jacket 440 and the conductors 446. In the embodiment of FIG. 4B, the RF shield 450 is disposed within the lead jacket 440 so that the RF shield is between a portion 440a of the lead jacket and the conductors 446. For example, the RF shield 450 can be coextruded with the lead jacket 440 or otherwise incorporated within the material of the lead jacket. In other embodiments, the lead jacket can be made of two separate pieces: a cover 440a and a liner 440b. The cover 440a and liner 440b can be made of two different materials or can be made of the same material.

In at least some embodiments, the multi-lumen conductor guide 442 includes the conductor lumens 446 arranged about the central lumen 448 such that there are no other lumens extending along the multi-lumen conductor guide between the central lumen and each of the multiple conductor lumens. In some embodiments, the conductor lumens 446 are each configured and arranged to receive a single conductor 444. In other embodiments, at least one of the conductor lumens is configured and arranged to receive multiple conductors. The multi-lumen conductor guide 442 may extend an entire longitudinal length of the lead 403 from the electrodes 134 (FIG. 1) to the terminals 310 (FIG. 3A). The conductor lumens 446 and central lumen 448 can have any suitable cross-sectional shape (e.g., round, oval, rectangular, triangular, or the like). The central lumen 448 and the plurality of conductor lumens 446 can be arranged in any suitable manner. In at least some embodiments, the conductor lumens 446 are disposed in the multi-lumen conductor guide 442 such that the conductor lumens 446 are peripheral to the central lumen 448.

A multi-lumen conductor guide 442 can be formed of any suitable material including, but not limited to, polyurethane, silicone, or silicone-polyurethane copolymer. It will be recognized that the multi-lumen conductor guide 442 need not have the specific form illustrated in FIG. 4 and that other conductor guide arrangements can be used including arrangements that permit more than one conductor per lumen or includes fewer conductor lumens (in some instances, a single conductor lumen). In some embodiments, the conductor guide 442 may be formed around the conductors 444 by molding or other methods. In some embodiments, the conductor guide 442 may be formed first and then the conductors 444 can be inserted into the conductor guide 442.

A variety of other arrangements of conductors can be utilized beyond those illustrated in FIGS. 4A and 4B. In particular, one or more of the conductors can be coiled around at least one of the other conductors. The coiled conductors provide additional RF shielding to the conductors within the coil as the coiled conductors form an inductor that resists rapidly changing electromagnetic fields (i.e., changing magnetic fields or RF irradiation or the like). Each of the arrangements of conductors illustrated in FIGS. 5-8 can be disposed within the jacket 440 and RF shield 450 of either embodiment of FIGS. 4A and 4B replacing of the multi-lumen conductor guide 442 and conductors 446 of those embodiments.

FIG. 5 illustrates another arrangement of conductors 544a, 544b with a multi-lumen conductor guide 542 having conductor lumens 546 and a central lumen 548. In the illustrated embodiment, one first conductor 544a is coiled around the multi-lumen conductor guide 542 with seven second conductors 544b extending along the conductor lumens 546 of the multi-lumen conductor guide. In other embodiments, more than one first conductor 554a (for example, two, three, four, five, six, or seven or more conductors) is coiled around the multi-lumen conductor guide that includes one or more second conductor 554b (for example, one, two, three, four, five, six, or seven or more conductors).

The second conductors 544b and associated conductor lumens 446 can extend straight relative to the lead body or can twist one or more times along the lead body or can be helically arranged along the lead body. The first conductor(s) 544a can have any suitable pitch (e.g., the center-to-center separation distance between successive coils) and the pitch can be the same along the entire lead or can be vary. In some embodiments, the pitch of the first conductor(s) 544a is selected so that there is little or no space between coils, as illustrated in FIG. 5.

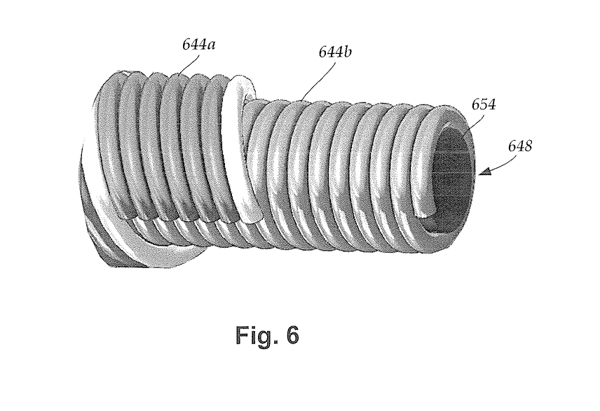

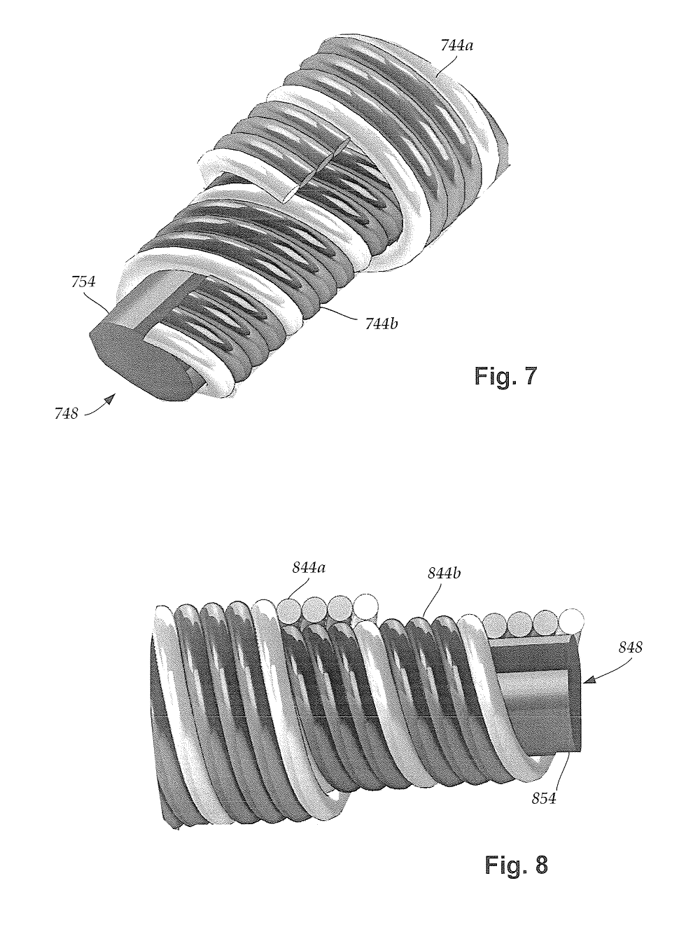

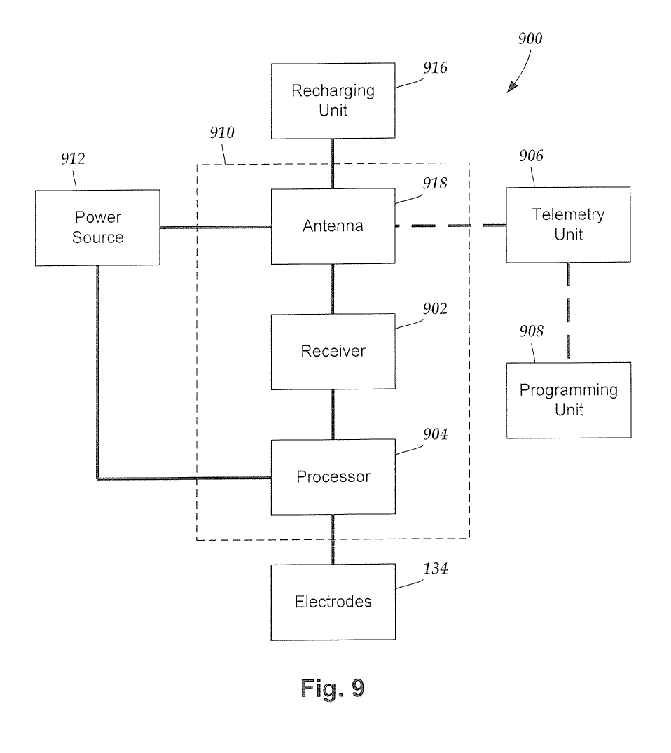

FIG. 6 illustrates another arrangement of conductors 644a, 644b disposed around stylet tube 654 (for example, a tube made of polyurethane, expanded polytetrafluoroethylene (ePTFE), or any other suitable material) with a central lumen 648. In the illustrated embodiment, seven first conductors 644a (one of which is shaded differently for illustration purposes) are coiled around a single second conductor 644b. In other embodiments, one or more first conductors 554a (for example, one, two, three, four, five, six, or seven or more conductors) are coiled around one or more second conductors 554b (for example, one, two, three, four, five, six, or seven or more conductors). For example, FIG. 7 illustrates an embodiment with four first conductors 744a (one of which is shaded differently for illustration purposes) coiled around four second conductors 744b (one of which is shaded differently for illustration purposes). FIG. 8 also illustrates another embodiment with four first conductors 844a (one of which is shaded differently for illustration purposes) coiled around four second conductors 844b (one of which is shaded differently for illustration purposes). In yet another embodiment, all of the conductors are coiled together in a single layer.

In some embodiments, such as the embodiments of FIGS. 6 and 8, the first conductors 644a, 844a are wound in the same direction as the second conductors 644b, 844b. In other embodiments, such as the embodiment of FIG. 7, the first conductors 744a are wound in a direction opposite the second conductors 744b.

The first conductor(s) and the second conductor(s) can have any suitable pitch (e.g., the center-to-center separation distance between successive coils) and the pitch can be the same along the entire lead or can vary. The pitch of the first conductor(s) can be the same or different from the second conductor(s). In some embodiments, the pitch of the first conductor(s) is selected so that there is little or no space between coils, as illustrated in FIGS. 6-8. In some embodiments, the pitch of the second conductor(s) is selected so that there is little or no space between coils, as illustrated in FIGS. 6-8.

The RF shield 450 (see, FIGS. 4A and 4B) can be disposed directly on the first conductor(s) in any of the arrangements illustrated in FIGS. 5-8 or can be disposed within the lead jacket 440 (see, FIG. 4B) that covers the first conductor(s) in any of the arrangements illustrated in FIGS. 5-8. In one embodiment, any of the arrangements illustrated in FIGS. 5-8 can be coextruded with the RF shield and lead jacket. In other embodiments, the RF shield and lead jacket can be slid, molded, or otherwise disposed over any of the arrangements illustrated in FIGS. 5-8.

In one example of a method of making a lead, any of the arrangements of first and second conductors is coiled over a stylet tube. The RF shield is then disposed over the first conductors. In some embodiments, one end of the RF shield may be fixed permanently or temporarily to prevent bunching of the shield during further manufacturing. The lead jacket can then be formed or otherwise disposed over the RF shield. In some embodiments, the lead jacket may be reflowed (e.g., heated to allow the polymeric material of the lead jacket to flow) into or around the RF shield. In some embodiments, small sections of the multi-lumen conductor guide may be provided on the proximal and distal ends of the lead to facilitate forming the arrays of terminals and electrodes (or other conductive contacts for the lead extension.) The conductors can be routed through respective conductor lumens with openings in the conductor lumens formed to allow coupling of the conductors to terminals, electrodes, or other conductive contacts disposed around the multi-lumen conductor guide.

FIG. 9 is a schematic overview of one embodiment of components of an electrical stimulation system 900 including an electronic subassembly 910 disposed within a control module. It will be understood that the electrical stimulation system can include more, fewer, or different components and can have a variety of different configurations including those configurations disclosed in the stimulator references cited herein.

Some of the components (for example, a power source 912, an antenna 918, a receiver 902, and a processor 904) of the electrical stimulation system can be positioned on one or more circuit boards or similar carriers within a sealed housing of an implantable pulse generator, if desired. Any power source 912 can be used including, for example, a battery such as a primary battery or a rechargeable battery. Examples of other power sources include super capacitors, nuclear or atomic batteries, mechanical resonators, infrared collectors, thermally-powered energy sources, flexural powered energy sources, bioenergy power sources, fuel cells, bioelectric cells, osmotic pressure pumps, and the like including the power sources described in U.S. Pat. No. 7,437,193, incorporated herein by reference.

As another alternative, power can be supplied by an external power source through inductive coupling via the optional antenna 918 or a secondary antenna. The external power source can be in a device that is mounted on the skin of the user or in a unit that is provided near the user on a permanent or periodic basis.

If the power source 912 is a rechargeable battery, the battery may be recharged using the optional antenna 918, if desired. Power can be provided to the battery for recharging by inductively coupling the battery through the antenna to a recharging unit 916 external to the user. Examples of such arrangements can be found in the references identified above.

In one embodiment, electrical current is emitted by the electrodes 134 on the paddle or lead body to stimulate nerve fibers, muscle fibers, or other body tissues near the electrical stimulation system. The processor 904 is generally included to control the timing and electrical characteristics of the electrical stimulation system. For example, the processor 904 can, if desired, control one or more of the timing, frequency, strength, duration, and waveform of the pulses. In addition, the processor 904 can select which electrodes can be used to provide stimulation, if desired. In some embodiments, the processor 904 selects which electrode(s) are cathodes and which electrode(s) are anodes. In some embodiments, the processor 904 is used to identify which electrodes provide the most useful stimulation of the desired tissue.

Any processor can be used and can be as simple as an electronic device that, for example, produces pulses at a regular interval or the processor can be capable of receiving and interpreting instructions from an external programming unit 908 that, for example, allows modification of pulse characteristics. In the illustrated embodiment, the processor 904 is coupled to a receiver 902 which, in turn, is coupled to the optional antenna 918. This allows the processor 904 to receive instructions from an external source to, for example, direct the pulse characteristics and the selection of electrodes, if desired.

In one embodiment, the antenna 918 is capable of receiving signals (e.g., RF signals) from an external telemetry unit 906 which is programmed by the programming unit 908. The programming unit 908 can be external to, or part of, the telemetry unit 906. The telemetry unit 906 can be a device that is worn on the skin of the user or can be carried by the user and can have a form similar to a pager, cellular phone, or remote control, if desired. As another alternative, the telemetry unit 906 may not be worn or carried by the user but may only be available at a home station or at a clinician's office. The programming unit 908 can be any unit that can provide information to the telemetry unit 906 for transmission to the electrical stimulation system 900. The programming unit 908 can be part of the telemetry unit 906 or can provide signals or information to the telemetry unit 906 via a wireless or wired connection. One example of a suitable programming unit is a computer operated by the user or clinician to send signals to the telemetry unit 906.

The signals sent to the processor 904 via the antenna 918 and the receiver 902 can be used to modify or otherwise direct the operation of the electrical stimulation system. For example, the signals may be used to modify the pulses of the electrical stimulation system such as modifying one or more of pulse duration, pulse frequency, pulse waveform, and pulse strength. The signals may also direct the electrical stimulation system 900 to cease operation, to start operation, to start charging the battery, or to stop charging the battery. In other embodiments, the stimulation system does not include the antenna 918 or receiver 902 and the processor 904 operates as programmed.

Optionally, the electrical stimulation system 900 may include a transmitter (not shown) coupled to the processor 904 and the antenna 918 for transmitting signals back to the telemetry unit 906 or another unit capable of receiving the signals. For example, the electrical stimulation system 900 may transmit signals indicating whether the electrical stimulation system 900 is operating properly or not or indicating when the battery needs to be charged or the level of charge remaining in the battery. The processor 904 may also be capable of transmitting information about the pulse characteristics so that a user or clinician can determine or verify the characteristics.

The above specification, examples and data provide a description of the manufacture and use of the composition of the invention. Since many embodiments of the invention can be made without departing from the spirit and scope of the invention, the invention also resides in the claims hereinafter appended.

What is claimed as new and desired to be protected by Letters Patent of the United States is:

1. An electrical stimulation lead, comprising:
at least one lead body having a distal end portion, a proximal end portion, and an outer surface, each of the at least one lead body comprising a lead jacket forming at least a portion of the outer surface of the lead body, the lead jacket comprising a non-conductive lead jacket material;
a plurality of electrodes disposed along the distal end portion of the at least one lead body;
a plurality of terminals disposed along the proximal end portion of the at least one lead body;
a plurality of conductors extending within the at least one lead body, each conductor of the plurality of conductors electrically coupling at least one of the plurality of terminals to at least one of the plurality of electrodes, wherein the plurality of conductors comprises at least one first conductor and at least one second conductor, wherein the at least one first conductor is coiled around the at least one second conductor; and
a conductive RF shield disposed between at least a portion of the lead jacket and around at least a portion of each of the plurality of conductors.

2. The electrical stimulation lead of claim 1, wherein the conductive RF shield is a conductive braided tube.

3. The electrical stimulation lead of claim 1, wherein the conductive RF shield is a conductive coiled tube.

4. The electrical stimulation lead of claim 1, wherein the conductive RF shield is configured and arranged to be electrically floating when the lead is used for electrical stimulation.

5. The electrical stimulation lead of claim 1, wherein the at least one first conductor is a single coiled first conductor and the at least one second conductor is a plurality of second conductors.

6. The electrical stimulation lead of claim 5, further comprising a multi-lumen conductor guide disposed within the lead body and within the single coiled first conductor, the multi-lumen conductor guide comprising a plurality of conductor lumens, wherein the plurality of second conductors are disposed within the conductor lumens.

7. The electrical stimulation lead of claim 6, wherein the plurality of second conductors extend straight relative to the lead body.

8. The electrical stimulation lead of claim 1, wherein the at least one first conductor is a plurality of first conductors cowound around the at least one second conductor.

9. The electrical stimulation lead of claim 8, wherein the at least one second conductor is coiled.

10. The electrical stimulation lead of claim 9, wherein the at least one first conductor and the at least one second conductor are coiled in a same direction.

11. The electrical stimulation lead of claim 9, wherein the at least one first conductor and the at least one second conductor are coiled in opposite directions.

12. The electrical stimulation lead of claim 1, wherein the RF shield extends between the plurality of terminals and the plurality of electrodes.

13. The electrical stimulation lead of claim 1, wherein the RF shield is disposed between the lead jacket and the plurality of conductors.

14. An electrical stimulating system comprising:
the electrical stimulation lead of claim 1; and
a control module coupleable to the electrical stimulation lead, the control module comprising
a housing, and
an electronic subassembly disposed in the housing.

15. The electrical stimulation system of claim 14, further comprising a lead extension coupleable to both the electrical stimulation lead and the control module.

16. A lead extension, comprising:
at least one body having a distal end portion, a proximal end portion, and an outer surface, each of the at least one body comprising a jacket forming at least a portion of the outer surface of the body, the jacket comprising a non-conductive jacket material;
a plurality of terminals disposed along the proximal end portion of the at least one body;
a connector disposed along the distal end portion of the at least one body, wherein the connector comprises a plurality of connector contacts and is configured and arranged to receive a portion of a lead;
a plurality of conductors extending within the at least one body from the plurality of connector contacts to the plurality of terminals, wherein the plurality of conductors are divided into one or more first conductors and one or more second conductors, wherein the one or more first conductors are coiled around the one or more second conductors; and
a conductive RF shield disposed between at least a portion of the jacket and around at least a portion of each of the plurality of conductors.

17. The lead extension of claim 16, wherein the conductive RF shield is a conductive braided tube.

18. The lead extension of claim 16, wherein the conductive RF shield is a conductive coiled tube.

19. The lead extension of claim 16, wherein the at least one first conductor is a plurality of first conductors cowound around the at least one second conductor.

20. The lead extension of claim 19, wherein the at least one second conductor is coiled.
